(19) United States
(12) Patent Application Publication
(10) Pub. No.: US 2010/0062073 A1
(43) Pub. Date: Mar. 11, 2010

(54) PHARMACEUTICAL COMPOSITIONS COMPRISING NANOPARTICLES COMPRISING ENTERIC POLYMERS CASEIN

(76) Inventors: Ronald Arthur Beyerinck, Bend, OR (US); Corey Jay Bloom, Bend, OR (US); Marshall David Crew, Bend, OR (US); Dwayne Thomas Friesen, Bend, OR (US); Michael Mark Morgen, Bend, OR (US); Daniel Tod Smithey, Bend, OR (US)

Correspondence Address: CHERNOFF, VILHAUER, MCCLUNG & STENZEL, LLP, 601 SW Second Avenue, Suite 1600, PORTLAND, OR, 97204-3157, US

(21) Appl. No.: 12/312,525

(22) PCT Filed: Nov. 16, 2007
(86) PCT No.: PCT/IB2007/003608

§ 371 Date: May 14, 2009

Related U.S. Application Data
(60) Provisional application No. 60/867,651, filed on Nov. 29, 2006.

(51) Int. Cl. A61K 9/51; A61P 43/00

(52) U.S. Cl. 424/491; 424/499

(57) ABSTRACT

A pharmaceutical composition comprises nanoparticles comprising a poorly water-soluble drug and an enteric polymer, and casein.

### BACKGROUND OF THE INVENTION

The present invention relates to compositions comprising nanoparticles comprising a low-solubility drug and an enteric polymer, and casein or a pharmaceutically acceptable form thereof.

It is known that poorly water-soluble drugs may be formulated as nanoparticles. Nanoparticles are of interest for a variety of reasons, such as to improve the bioavailability of poorly water-soluble drugs, to provide targeted drug delivery to specific areas of the body, to reduce side effects, or to reduce variability in vivo.

A variety of approaches have been taken to formulate drugs as nanoparticles. One approach is to decrease the size of a crystalline drug by grinding or milling the drug in the presence of a surface modifier. See, e.g., U.S. Pat. No. 5,145,684. Another approach to forming nanoparticles is to precipitate the drug in the presence of a film forming material such as a polymer. See, e.g., U.S. Pat. No. 5,118,528.

There remain a number of problems associated with the use of nanoparticles to deliver pharmaceutical compounds to the body. The nanoparticles must be stabilized so that they do not aggregate into larger particles in aqueous suspensions. Often surface modifiers such as surfactants are used to stabilize the nanoparticles, but such materials can have adverse physiological effects when administered in vivo. In addition, without a surface modifier present, the surface of the nanoparticles is unprotected, leading to a decrease in performance and stability. Additionally, when formulated as a dry material, the composition should spontaneously form nanoparticles when the composition is added to an aqueous use environment.

Casein has been used as a protective colloid for xanthophylls and other actives. See U.S. Pat. No. 6,863,914 and published U.S. Patent Application No. 2002/0110599A1. Casein has also been included in a long list of surface stabilizers for crystalline and amorphous cyclosporine nanoparticles. See U.S. Pat. No. 6,656,504. Casein has also been used as a protective coating for particles containing a therapeutic agent and a core comprising calcium phosphate. See published U.S. Patent Application No. 2002/0054914A1. Casein has also been used as a crosslinked matrix for nanoparticles. See U.S. Pat. No. 4,107,288. However, nanoparticles formed from a poorly water soluble drug and casein alone do not adequately solve the problems described above.

Accordingly, there is still a continuing need for nanoparticles that are stable, in the sense of not aggregating into larger particles, and that improve the bioavailability of low-solubility drugs.

### BRIEF SUMMARY OF THE INVENTION

In one aspect, a solid pharmaceutical composition comprises: (a) nanoparticles comprising a poorly water-soluble drug and an enteric polymer, wherein (i) the poorly water soluble drug has an aqueous solubility of less than 5 mg/mL over the pH range of 6.5 to 7.5; (ii) at least 90 wt % of the drug in the nanoparticles is in a non-crystalline form; (iii) the nanoparticles having an average size of less than 500 nm; and (iv) a mass ratio of the poorly water soluble drug to the enteric polymer is less than 9:1; and (b) casein or a pharmaceutically acceptable form thereof; wherein a mass ratio of (1) the casein to (2) the combined mass of the poorly water soluble drug and enteric polymer is at least 1:20.

In one embodiment, the casein is present in the nanoparticles. In another embodiment, the solid composition comprises a plurality of nanoparticles in a casein matrix. In still another embodiment, the solid composition comprises nanoparticles in a casein matrix wherein casein is also present in the nanoparticles.

In another aspect, a pharmaceutical composition comprises an aqueous suspension, the aqueous suspension comprising: (a) nanoparticles comprising a poorly water soluble drug and an enteric polymer, wherein (i) the poorly water soluble drug has an aqueous solubility of less than 5 mg/mL over the pH range of 6.5 to 7.5; (ii) at least 90 wt % of the drug in the nanoparticles is in a non-crystalline form; (iii) the nanoparticles have an average size of less than 500 nm; (iv) the poorly water soluble drug and the enteric polymer constitute at least 60 wt % of the nanoparticles; and (v) a mass ratio of the poorly water soluble drug to the enteric polymer is less than 9:1; (b) casein or a pharmaceutically acceptable form thereof; and (c) water.

The compositions of the present invention provide a number of advantages over the prior art. Because the pharmaceutical composition comprises (a) nanoparticles comprising a poorly water soluble drug and an enteric polymer, and (b) casein, the stability of the non-crystalline drug in the nanoparticles and the suspension/resuspension stability of the nanoparticles can be addressed independently, resulting in nanoparticles with improved performance and stability.

First, the enteric polymer used in the nanoparticles helps stabilize the poorly water soluble drug. The enteric polymer is chosen so that a portion of the drug is soluble in the enteric polymer. This helps prevent or reduce the rate of crystallization of the non-crystalline drug in the nanoparticle.

Second, the casein helps promote stability of aqueous suspensions of the nanoparticles, reducing, slowing, or preventing agglomeration of the nanoparticles. The use of casein also improves the re-suspendability of solid compositions containing nanoparticles relative to surfactant-based and ionizable polymer-based stabilizers: solid compositions of the invention resuspend nanoparticles when administered to an aqueous solution.

Finally, the nanoparticles of the invention may provide improved toleration relative to conventional nanoparticles that incorporate a substantial amount of a surfactant to stabilize the nanoparticles.

The foregoing and other objectives, features, and advantages of the invention will be more readily understood upon consideration of the following detailed description of the invention.

### BRIEF DESCRIPTION OF THE SEVERAL VIEWS OF THE DRAWINGS

FIG. 1. shows schematically a solid composition of the present invention.

### DETAILED DESCRIPTION OF THE INVENTION

The compositions of the present invention relate to (a) a plurality of nanoparticles, each of the nanoparticles comprising the drug and the enteric polymer, and (b) casein. Pharmaceutical compositions, nanoparticles, enteric polymers, casein, drugs, optional surface stabilizers, and methods for making nanoparticles and the compositions are described in detail below.

### Solid Pharmaceutical Compositions

In one aspect, the invention comprises a solid pharmaceutical composition comprising (a) a plurality of nanoparticles comprising a poorly water-soluble drug and an enteric polymer, and (b) casein or a pharmaceutically acceptable form thereof. As used herein, the term “solid pharmaceutical composition” means that the composition is in a solid form and substantially free of liquids. Exemplary forms for the solid pharmaceutical composition include particles, granules, powders, dust, pellets, flakes, slabs, rods, and tablets. Methods for making such solid compositions are described herein below.

By “nanoparticles” is meant a plurality of small particles in which the average size of the particles less than about 500 nm. In suspension, by “average size” is meant the effective cumulant diameter as measured by dynamic light scattering, using for example, Brookhaven Instruments' 90Plus particle sizing instrument. By “size” is meant the diameter for spherical particles, or the maximum diameter for non-spherical particles. Preferably, the average size of the nanoparticles is less than 400 nm, more preferably less 300 nm, and most preferably less than 200 nm.

The width of the particle size distribution in suspension is given by the “polydispersity” of the particles, which is defined as the relative variance in the correlation decay rate distribution, as is known by one skilled in the art. See B. J. Fisken, “Revisiting the method of cumulants for the analysis of dynamic light-scattering data,” Applied Optics, 40(24), 4087-4091 (2001) for a discussion of cumulant diameter and polydispersity. Preferably, the polydispersity of the nanoparticles is less than 0.5. More preferably, the polydispersity of the nanoparticles is less than about 0.3. In one embodiment, the average size of the nanoparticles is less than 500 nm with a polydispersity of 0.5 or less. In another embodiment, the average size of the nanoparticles is less than 300 nm with a polydispersity of 0.5 or less.

In one embodiment, the casein is present in the nanoparticles together with the poorly water-soluble drug and the enteric polymer. In this embodiment, the casein may act as a surface stabilizer, stabilizing the nanoparticles during the formation process or when present in aqueous suspension, reducing or preventing aggregation or flocculation of the nanoparticles.

In another embodiment, the solid compositions comprise a plurality of nanoparticles in a casein matrix. By “casein matrix” is meant that at least a portion of the nanoparticles in the solid composition are encapsulated by the casein. By “at least a portion of the nanoparticles are encapsulated by the casein” means that the casein encapsulates at least a portion of the plurality of nanoparticles in the composition. The casein may encapsulate only a portion of nanoparticles, or may encapsulate essentially all of the nanoparticles in the composition.

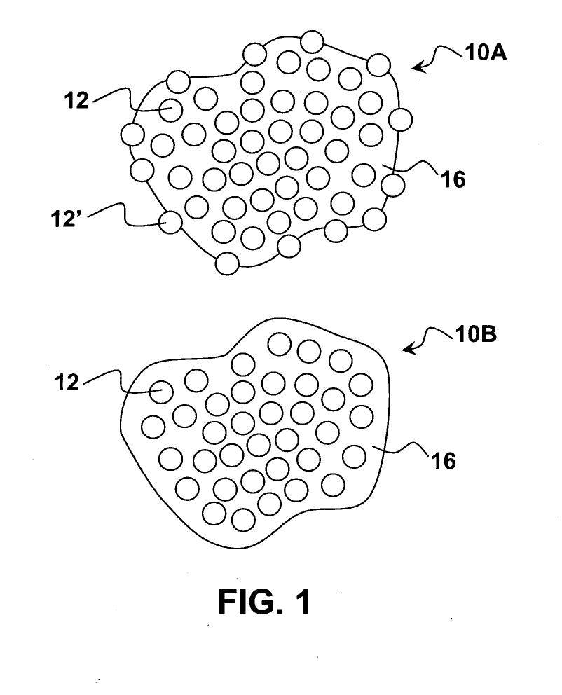

For example, FIG. 1 shows schematically a composition 10A comprising nanoparticles 12 encapsulated by the casein 16. Those nanoparticles 12′ not encapsulated by the casein 16 have at least a portion of their surfaces in contact with the casein 16. Composition 10B has essentially all of the nanoparticles 12 encapsulated with the casein 16. Thus, the compositions may contain a plurality of nanoparticles, at least a portion of which are encapsulated by the casein.

For compositions comprising nanoparticles in a casein matrix, the presence of nanoparticles in the solid composition can be determined using the following procedure. A sample of the solid composition is embedded in a suitable material, such as an epoxy or polyacrylic acid (e.g., LR White from London Resin Co., London, England). The sample is then microtomed to obtain a cross-section of the solid composition that is about 100 to 200 nm thick. This sample is then analyzed using transmission electron microscopy (TEM) with energy dispersive X-ray (EDX) analysis. TEM-EDX analysis quantitatively measures the concentration and type of atoms larger than boron over the surface of the sample. From this analysis, regions that are rich in drug and enteric polymer can be distinguished from regions that are rich in casein. The size of the regions that are rich in drug and polymer will have an average diameter of less than 500 nm in this analysis, demonstrating that the solid composition comprises nanoparticles of drug and enteric polymer, and casein. See, for example, Transmission Electron Microscopy and Diffractometry of Materials (2001) for further details of the TEM-EDX method.

Another procedure that demonstrates the solid composition contains nanoparticles is to administer a sample of the solid composition to water to form a suspension of the nanoparticles. The suspension is then analyzed by dynamic light scattering (DLS) as described herein below. A solid composition of the invention will form nanoparticles having an average cumulant diameter of less than 500 nm.

A specific procedure for demonstrating the solid composition contains nanoparticles is as follows. A sample of the solid composition is added to water at ambient temperature such that the concentration of solids is less than about 1 mg/mL. The so-formed suspension is then analyzed by DLS. The solid composition contains nanoparticles if the DLS analysis results in particles having an average cumulant diameter of less than 500 nm.

A solid composition of the invention will show the presence of nanoparticles in at least one, and preferably both of the above tests.

Generally, it is preferred that the solid compositions of the present invention be in the form of small particles or a powder. The small particles or powder may be formed in the process of making the solid composition, or may be formed subsequent to formation of the solid composition. Processes for preparing the compositions of the present invention are discussed herein below.

Preferably, the mean diameter of the small particles of the composition of the present invention will range from about 1 μm to about 500 μm. For improved processing of the solid composition, larger particles are generally preferred. Thus, the mean diameter of the particles is preferably at least 5 μm, more preferably at least 10 μm, or even more preferably at least 25 μm. However, if the particles are too large, the rate of disintegration of the particles can be affected. Thus, the mean diameter may be less than 500 μm, or less than 100 μm in diameter. The mean diameter of the particles preferably ranges from 10 μm to 500 μm, more preferably from 25 μm to 100 μm.

The nanoparticles and casein are collectively present in the solid composition in an amount ranging from about 60 wt % to 100 wt % of the total mass of the composition. Preferably, the nanoparticles and the casein collectively constitute at least 70 wt %, more preferably at least 80 wt %, and even more preferably at least 90 wt % of the composition. In one embodiment, the composition consists essentially of the nanoparticles and the casein. By “consists essentially of” is meant that the composition contains less than 1 wt % of any other excipients and that any such excipients have no affect on the performance or properties of the composition.

The mass ratio of the casein to the mass of the nanoparticles in the composition may range from 1:20 to about 9:1. The casein is preferably present in a sufficient amount so that the nanoparticles re-suspend when the solid composition is administered to an aqueous use environment. Furthermore, preferably a sufficient amount of casein is present to prevent or retard agglomeration of the nanoparticles into larger particles following administration to an aqueous use environment. Thus, the mass ratio of the casein to nanoparticles is at least about 1:20, more preferably at least about 1:15, more preferably at least about 1:10, more preferably at least about 1:7, more preferably at least about 1:5, and most preferably at least about 1:4.

In a preferred embodiment, the solid composition of the present invention has the following composition relative to the total mass of drug, enteric polymer, and casein in the composition:

1 to 60 wt % drug;

10 to 80 wt % enteric polymer; and

5 to 50 wt % casein.

In another embodiment, the invention comprises an aqueous suspension comprising a plurality of nanoparticles, casein, and water. Preferably, the casein is associated with the nanoparticles in the suspension. By “associated with” is meant that a portion of the casein in the suspension is in contact with or is adsorbed to the surface of the nanoparticles.

Suspensions comprising the nanoparticles, casein, and water may be formed by administering the solid pharmaceutical compositions described above to water or other appropriate aqueous solution. Alternatively, the suspensions may be formed by forming the nanoparticles in an aqueous solution and adding casein. In yet another method, the suspensions may be formed by forming the nanoparticles in an aqueous solution containing casein. These and other methods for forming suspensions of the present invention are described herein below.

### Nanoparticles

The compositions of the present invention comprise a plurality of nanoparticles, each of the nanoparticles comprising the drug and the enteric polymer. While the drug in its pure form may be either crystalline or non-crystalline, at least 90 wt % of the drug in the nanoparticles is non-crystalline. The term “crystalline,” as used herein, means a particular solid form of a compound that exhibits long-range order in three dimensions. “Non-crystalline” refers to material that does not have long-range three-dimensional order, and is intended to include not only material which has essentially no order, but also material which may have some small degree of order, but the order is in less than three dimensions and/or is only over short distances. Another term for a non-crystalline form of a material is the “amorphous” form of the material. It has been found that for poorly water-soluble drugs having poor bioavailability that bioavailability improves as the fraction of drug present in the non-crystalline state in the nanoparticle increases. Preferably at least about 95 wt % of the drug in the nanoparticle is non-crystalline; in other words, the amount of drug in crystalline form does not exceed about 5 wt %. Amounts of crystalline drug may be measured by Powder X-Ray Diffraction (PXRD), by Differential Scanning Calorimetry (DSC), by solid-state nuclear magnetic resonance (NMR), or by any other known quantitative measurement.

The non-crystalline drug in the nanoparticle can exist as a pure phase, as a solid solution of drug homogeneously distributed throughout the enteric polymer, or any combination of these states or those states that lie between them. Preferably, at least a portion of the drug and the enteric polymer is present in the nanoparticle in the form of a solid solution. The solid solution may be thermodynamically stable, in which the drug is present at less than the solubility limit of the drug in the enteric polymer, or may be a supersaturated solid solution in which the drug exceeds its solubility limit in the enteric polymer. Preferably essentially all of the drug and the enteric polymer is present as a solid solution.

In one embodiment, the nanoparticles comprise a core, the core comprising the non-crystalline drug and the enteric polymer. As used herein, the term “core” refers to the central portion of the nanoparticle. In some embodiments, described herein below, materials may be adsorbed to the surface of the core. Materials adsorbed to the surface of the core are considered part of the nanoparticle, but are distinguishable from the core of the nanoparticle. Methods to distinguish materials present in the core versus materials adsorbed to the surface of the core include (1) thermal methods, such as differential scanning calorimetry (DSC); (2) spectroscopic methods, such as X-ray photoelectron spectroscopy (XPS), transmission electron microscopy (TEM) with energy dispersive X-ray (EDX) analysis, Fourier transform infra red (FTIR) analysis, and Raman spectroscopy; (3) chromatographic techniques, such as high performance liquid chromatography (HPLC), and gel-permeation chromatography (GPC); and (4) other techniques known in the art.

In one embodiment, the non-crystalline drug and the enteric polymer constitute at least 60 wt % of the core, more preferably at least 80 wt % of the core. In another embodiment, the core consists essentially of the non-crystalline drug and the enteric polymer.

The non-crystalline drug present in the core can exist in non-crystalline pure drug domains, as a thermodynamically stable solid solution of non-crystalline drug homogeneously distributed throughout the enteric polymer, as a supersaturated solid solution of non-crystalline drug homogeneously distributed throughout the enteric polymer, or any combination of these states or those states that lie between them. When the glass-transition temperature (T_{g}) of the non-crystalline drug is different from the T_{g} of the pure polymer by at least about 20° C., the core may exhibit a T_{g} that is between the T_{g} of pure non-crystalline drug or pure polymer. Preferably, less than 20 wt % of the drug is present in non-crystalline drug domains, with the remaining drug homogeneously distributed throughout the enteric polymer.

In yet another embodiment, the core comprises the non-crystalline drug, the enteric polymer, and casein or a pharmaceutically acceptable form thereof. The core may be (1) a homogeneous molecular mixture of drug, enteric polymer, and casein, (2) domains of pure drug, domains of pure enteric polymer, and domains of pure casein distributed throughout the core, or (3) any combination of these states or those states that lie between them. In one embodiment, the drug, enteric polymer, and casein are homogeneously distributed throughout the core as a supersaturated solid solution. In another embodiment, the exterior surface of the core has a higher concentration of casein relative to the core as a whole.

In still another embodiment, the core comprises the non-crystalline drug and the enteric polymer, with the casein adsorbed to the surface of the core.

In yet another embodiment, the core comprises the non-crystalline drug, the enteric polymer, and a portion of the casein. The remaining portion of the casein is adsorbed to the surface of the core. In this embodiment, a portion of the casein is integral to the core, while the remaining portion of casein is adsorbed to the surface of the core.

The mass ratio of drug to enteric polymer in the nanoparticle can range from about 1:999 to about 9:1 (that is, from about 0.1 wt % drug to 90 wt % drug relative to the total mass of drug and enteric polymer in the nanoparticle). Preferably, the mass ratio of drug to enteric polymer ranges from about 1:99 to about 4:1 (that is, from about 1 wt % to about 80 wt % drug relative to the total mass of drug and enteric polymer), more preferably from about 1:19 to about 3:1 (that is, from about 5 wt % to about 75 wt %), even more preferably from about 1:9 to about 2:1 (that is, from about 10 wt % to about 67 wt % drug relative to the total mass of drug and enteric polymer in the nanoparticle), and most preferably from about 1:3 to about 3:2 (that is, from about 25 wt % to about 60 wt % drug relative to the total mass of drug and enteric polymer in the nanoparticle). In one embodiment, the mass ratio of drug to enteric polymer is less than 9:1, preferably less than 4:1, more preferably less than 3:1, and most preferably less than 3:2. In another embodiment, the mass ratio of drug to enteric polymer is at least 1:999, preferably at least 1:99, more preferably at least 1:9, and most preferably at least 1:3.

To minimize the total mass of the formulation, high drug loadings are desired. However, if the amount of drug in the nanoparticle is too high, the nanoparticles can become unstable. This can lead to (1) crystallization of the drug in the nanoparticle, and/or (2) phase separation of the drug in the nanoparticle, both of which lead to a non-homogeneous composition. In absolute terms, it is generally preferred that the amount of drug in the nanoparticle be less than about 90 wt %, more preferably less than about 80 wt %, even more preferably less than about 75 wt % the total mass of the nanoparticle.

### Enteric Polymers

The term “polymer” is used conventionally, meaning a compound that is made of monomers connected together to form a larger molecule. A polymer generally consists of at least about 20 monomers connected together. Thus, the molecular weight of the polymer generally will be about 2000 daltons or more. The polymer should be inert, in the sense that it does not chemically react with the drug in an adverse manner, and should be pharmaceutically acceptable.

The polymer is an “enteric polymer,” meaning that the polymer is poorly soluble in water at a pH of about 4.5 or less, but is soluble in water at a pH of greater than about 5. The term “poorly soluble” as used in connection with enteric polymers herein refers to a solubility of less than about 0.1 mg/mL or less when administered at a concentration of 0.2 mg/mL to water having a pH of about 4.5 or less. Enteric polymers have at least one ionizable substituent that is capable of being ionized at a pH of greater than about 5. Enteric polymers are typically polyacids having a pKa of about 3 to 6. Exemplary ionizable substituents include carboxylic acids, thiocarboxylic acids, and sulfonates. Preferred ionizable substituents include ether-lined alkyl sulfonates such as ethyl sulfonates, ether-linked alkyl carboxy groups, such as carboxy methyl and carboxy ethyl, and ester-linked substituents comprising a carboxylic acid group such as succinate, phthalate, trimellitate, and maleate. The number of ionizable groups covalently attached to the polymer is preferably at least about 0.05 milliequivalents per gram of polymer. Preferably, the number is at least about 0.1 milliequivalents per gram of polymer.

At a pH of greater than about 5, the enteric polymer is aqueous soluble. By “aqueous soluble” is meant that when the polymer is administered alone at a solids concentration of 0.2 mg/mL to a phosphate buffered saline (PBS) solution consisting of an aqueous solution of 20 mM sodium phosphate (Na₂HPO₄), 47 mM potassium phosphate (KH₂PO₄), 87 mM NaCl, and 0.2 mM KCl, adjusted to pH 6.5 with NaOH, the polymer has a solubility of greater than 0.1 mg/mL. Preferably, the polymer has a solubility of at least 0.13 mg/mL, more preferably at least 0.15 mg/mL, and most preferably at least 0.17 mg/mL.

It is also preferred that the enteric polymer be soluble in an organic solvent. Preferably the enteric polymer has a solubility in an organic solvent of at least about 0.1 mg/mL, and preferably at least 1 mg/mL. Preferably the enteric polymer is not crosslinked.

The enteric polymer may also have a high glass-transition temperature (T_{g}). By “high glass-transition temperature” is meant that the T_{g} of the enteric polymer is at least 50° C. when measured at a relative humidity (RH) of 75% or more. Preferably, the T_{g} of the enteric polymer is at least 60° C., more preferably at least 70° C., when measured at an RH of 75% or more.

Suitable enteric polymers include substituted polysaccharides, and non-polysaccharides. By substituted polysaccharides is meant that the enteric polymer has a polysaccharide backbone that has been modified by reaction of at least a portion of the hydroxyl groups on the saccharide repeating units with a compound to form an ester or an ether substituent. Exemplary polysaccharide backbone polymers include cellulose, starch, dextran, dextrin, amylose, amylose pectin, and pullulan.

In one embodiment, the substituted polysaccharide enteric polymer is a cellulosic polymer. By “cellulosic” is meant a cellulose polymer that has been modified by reaction of at least a portion of the hydroxyl groups on the saccharide repeating units with a compound to form an ester or an ether substituent.

Exemplary enteric cellulosic polymers include: hydroxypropyl methyl cellulose acetate succinate, hydroxypropyl methyl cellulose phthalate, carboxymethyl ethylcellulose, cellulose acetate phthalate, cellulose acetate succinate, hydroxypropyl methyl cellulose acetate phthalate, cellulose acetate trimellitate, hydroxypropyl methyl cellulose acetate trimellitate, and mixtures thereof.

In another embodiment, the enteric polymer is a non-polysaccharide polymer. Exemplary non-polysaccharide enteric polymers include vinyl polymers, such as polyvinyl acetate phthalate, vinyl acetate-maleic anhydride copolymer; polyacrylates, polymethacrylates, and copolymers thereof, such as methyl acrylate-methacrylic acid copolymer, ethyl acrylate-methacrylic acid copolymers; styrene-maleic acid copolymers; shellac, and mixtures thereof.

In one embodiment, the enteric polymer is selected from the group consisting of hydroxypropyl methyl cellulose acetate succinate, hydroxypropyl methyl cellulose phthalate, carboxymethylethyl cellulose, cellulose acetate phthalate, cellulose acetate succinate, hydroxypropyl methyl cellulose acetate phthalate, cellulose acetate trimellitate, hydroxypropyl methyl cellulose acetate trimellitate, polyvinyl acetate phthalate, vinyl acetate-maleic anhydride copolymer, polyacrylates, methyl acrylate-methacrylic acid copolymers, ethyl acrylate-methacrylic acid copolymers, styrene-maleic acid copolymers, shellac, and mixtures thereof.

In another embodiment, the enteric polymer is selected from the group consisting of hydroxypropyl methyl cellulose acetate succinate, carboxymethylethyl cellulose, hydroxypropyl methyl cellulose phthalate, cellulose acetate phthalate, cellulose acetate trimellitate, methyl acrylate-methacrylic acid copolymers, ethyl acrylate-methacrylic acid copolymers, and mixtures thereof.

### Surface Stabilizers

The nanoparticles of the present invention may optionally comprise a surface stabilizer in addition to the drug and the enteric polymer. The purpose of the surface stabilizer is to reduce or prevent aggregation or flocculation of the nanoparticles in an aqueous suspension, resulting in nanoparticles with improved stability. In one embodiment, the surface stabilizer is used to stabilize the nanoparticles during the formation process. The stabilizer should be inert, in the sense that it does not chemically react with the drug in an adverse manner, and should be pharmaceutically acceptable.

The optional surface stabilizer may constitute from 0 wt % to about 40 wt % of the total mass of the nanoparticles. Generally, lower concentrations of surface stabilizer are preferred. Thus, preferably the surface stabilizer constitutes about 35 wt % or less, more preferably about 30 wt % or less, and most preferably about 25 wt % or less the total mass of the nanoparticles.

In one embodiment, the poorly water soluble drug, the enteric polymer, the optional surface stabilizer, and the casein constitute at least 90 wt % of the solid composition of the invention. In another embodiment, the solid composition of the invention consists essentially of the poorly water soluble drug, the enteric polymer, the optional surface stabilizer, and the casein.

In one embodiment, the surface stabilizer is an amphiphilic compound, meaning that it has both hydrophobic and hydrophilic regions. In another embodiment, the surface stabilizer is a surfactant, including anionic, cationic, zwitterionic, and non-ionic surfactants. Mixtures of surface stabilizers may also be used.

Exemplary surface stabilizers include casein, caseinates, polyvinyl pyrrolidone (PVP), polyoxyethylene alkyl ethers, polyoxyethylene stearates, polyoxyethylene castor oil derivatives, poly(ethylene oxide-propylene oxide) (also known as poloxamers), tragacanth, gelatin, polyethylene glycol, bile salts (such as salts of dihydroxy cholic acids, including sodium and potassium salts of cholic acid, glycocholic acid, and taurocholic acid), phospholipids (such as phosphatidyl cholines, including 1,2-diacylphosphatidylcholine also referred to as PPC or lecithin), sodium dodecylsulfate (also known as sodium lauryl sulfate), benzalkonium chloride, sorbitan esters, polyoxyethylene alkyl ethers, polyoxyethylene castor oil derivatives, polyoxyethylene sorbitan fatty acid esters (polysorbates), polyoxyethylene stearates, triethanolamine, sodium docusate, sodium stearyl fumarate, sodium cyclamate, and mixtures and pharmaceutically acceptable forms thereof.

When casein is used as a surface stabilizer, the casein may be present during the formation of the nanoparticles, or added following formation of the nanoparticles, as discussed herein below. The amount of casein required to stabilize the nanoparticles should generally be at least 5 wt % of the total mass of the nanoparticles, preferably at least 10 wt % of the nanoparticles. When casein is used as a surface stabilizer, additional casein may be included in the composition such that the nanoparticles are present in a casein matrix, as described herein above.

### Casein

The compositions of the present invention also comprise casein or a pharmaceutically acceptable form thereof. As used herein, the term “casein” refers to phosphoproteins occurring in milk, cheese, and other natural products. The term casein also includes so-called vegetable caseins, also known as legumin or avenin. Vegetable caseins are found in beans and nuts, and are globulin proteins resembling caseins present in milk. Caseins are small proteins with molecular weights ranging from about 10,000 Daltons to about 50,000 Daltons. The casein content of bovine milk represents about 80% of milk proteins, while caseins represent only about 40% of the protein in human milk. Caseins are typically obtained from milk by precipitation at pH 4.6 at 20° C. Under these conditions, the proteins that precipitate are called caseins. There are four main proteins in bovine casein: αₛ₁-casein, αₛ₂-casein, β-casein, and κ-casein.

The caseins are amphiphilic, possessing relatively hydrophobic regions and relatively hydrophilic regions. As a result, caseins are highly surface active. Caseins are sparingly soluble in water, and typically exist in a colloidal particle known as a casein micelle. It is believed that κ-casein is located on the surface of the micelle and contributes to the stability and structure of the micelle. See for example Proteins in Food Processing, (Chapter 3, “The Caseins,” P. F. Fox and A. L. Kelly, Woodhead Publishing Limited, 2004).

As used herein, by “a pharmaceutically acceptable form thereof” is meant either an acid or base addition salt of casein. One preferred form of casein is caseinates. “Caseinates” are produced by reaction of casein with an alkaline substance. Exemplary caseinates include sodium caseinate, calcium caseinate, potassium caseinate and ammonium caseinate.

In one embodiment, the casein is a mixture of caseins found in milk. In another embodiment, the casein is a mixture of caseins found in bovine milk. In still another embodiment, the casein is αₛ₁-casein. In still another embodiment, the casein is αₛ₂-casein. In still another embodiment, the casein is β-casein. In yet another embodiment, the casein is κ-casein. In yet another embodiment, the casein is present as a pharmaceutically acceptable salt form, such as sodium caseinate, calcium caseinate, potassium caseinate or ammonium caseinate. In still another embodiment, the casein is selected from the group consisting of αₛ₁-casein, αₛ₂-casein, β-casein, κ-casein, vegetable casein, sodium caseinate, calcium caseinate, potassium caseinate, ammonium caseinate, and mixtures thereof.

### The Drug

The drug is a “poorly water soluble drug,” meaning that the drug has a solubility in water (over the pH range of 6.5 to 7.5 at 25° C.) of less than 5 mg/mL. The utility of the invention increases as the water solubility of the drug decreases. The drug may have an even lower solubility in water, such as less than about 1 mg/mL, less than about 0.1 mg/mL, and even less than about 0.01 mg/mL.

In general, it may be said that the drug has a dose-to-aqueous solubility ratio greater than about 10 mL, and more typically greater than about 100 mL, where the aqueous solubility (mg/mL) is the minimum value observed in any physiologically relevant aqueous solution (i.e., solutions with pH 1-8), including USP simulated gastric and intestinal buffers, and dose is in mg. Thus, a dose-to-aqueous solubility ratio may be calculated by dividing the dose (in mg) by the aqueous solubility (in mg/mL).

Preferred classes of drugs include, but are not limited to, antihypertensives, antianxiety agents, anticlotting agents, anticonvulsants, blood glucose-lowering agents, decongestants, antihistamines, antitussives, antineoplastics, beta blockers, anti-inflammatories, antipsychotic agents, cognitive enhancers, anti-atherosclerotic agents, cholesterol-reducing agents, triglyceride-reducing agents, antiobesity agents, autoimmune disorder agents, anti-impotence agents, antibacterial and antifungal agents, hypnotic agents, anti-Parkinsonism agents, anti-Alzheimer's disease agents, antibiotics, anti-depressants, antiviral agents, glycogen phosphorylase inhibitors, cholesteryl ester transfer protein (CETP) inhibitors, microsomal triglyceride transfer protein (MTP) inhibitors, anti-angiogenesis agents, vascular endothelial growth factor (VEGF) receptor inhibitors, and carbonic anhydrase inhibitors.

Each named drug should be understood to include the neutral form of the drug or pharmaceutically acceptable forms of the drug. By “pharmaceutically acceptable forms” is meant any pharmaceutically acceptable derivative or variation, including stereoisomers, stereoisomer mixtures, enantiomers, solvates, hydrates, isomorphs, polymorphs, pseudomorphs, neutral forms, salt forms and prodrugs.

Exemplary drugs suitable for use in the nanoparticles include sildenafil and sildenafil citrate, atorvastatin, lovastatin, simvastatin, pravastatin, fluvastatin, rosuvastatin, itavastatin, nisvastatin, visastatin, atavastatin, bervastatin, compactin, dihydrocompactin, dalvastatin, fluindostatin, pitivastatin, mevastatin, velostatin (also referred to as synvinolin), valdecoxib, celecoxib, torcetrapib, ziprasidone, and nifedipine. Other low-solubility drugs suitable for use in the nanoparticles are disclosed in US Published patent application 2005/0031692, herein incorporated by reference.

In one embodiment the drug is a cholesteryl ester transfer protein (CETP) inhibitor. CETP inhibitors are drugs that inhibit CETP activity. The effect of a drug on the activity of CETP can be determined by measuring the relative transfer ratio of radiolabeled lipids between lipoprotein fractions, essentially as previously described by Morton in J. Biol. Chem. 256, 11992, 1981 and by Dias in Clin. Chem. 34, 2322, 1988, and as presented in U.S. Pat. No. 6,197,786, the disclosures of which are herein incorporated by reference. The potency of CETP inhibitors may be determined by performing the above-described assay in the presence of varying concentrations of the test compounds and determining the concentration required for 50% inhibition of transfer of radiolabeled lipids between lipoprotein fractions. This value is defined as the “IC₅₀ value.” Preferably, the CETP inhibitor has an IC₅₀ value of less than about 2000 nM, more preferably less than about 1500 nM, even more preferably less than about 1000 nM, and most preferably less than about 500 nM.

Specific examples of CETP inhibitors include [2R,4S] 4-[(3,5-bis-trifluoromethyl-benzyl)methoxycarbonyl-amino]-2-ethyl-6-trifluoromethyl-3,4-dihydro-2H-quinoline-1-carboxylic acid ethyl ester (torcetrapib); [2R,4S] 4-[acetyl-(3,5-bis-trifluoromethyl-benzyl)-amino]-2-ethyl-6-trifluoromethyl-3,4-dihydro-2H-quinoline-1-carboxylic acid isopropyl ester; [2R, 4S] 4-[(3,5-bis-trifluoromethyl-benzyl)-methoxycarbonyl-amino]-2-ethyl-6-trifluoromethyl-3,4-dihydro-2H-quinoline-1-carboxylic acid isopropyl ester; (2R)-3-[[3-(4-chloro-3-ethylphenoxy)phenyl][[3-(1,1,2,2-tetrafluoroethoxy)phenyl]methyl]amino]-1,1,1-trifluoro-2-propanol; (2R, 4R, 4aS)-4-[amino-(3,5-bis-(trifluoromethyl-phenyl)-methyl]-2-ethyl-6-(trifluoromethyl)-3,4-dihydroquinoline-1-carboxylic acid isopropyl ester; S-[2-([[1-(2-ethylbutyl)cyclohexyl]carbonyl]amino)phenyl]2-methylpropanethioate; trans-4-[[[2-[[[[3,5-bis(trifluoromethyl)phenyl]methyl](2-methyl-2H-tetrazol-5-yl)amino]methyl]-4-(trifluoromethyl)phenyl]ethylamino]methyl]-cyclohexaneacetic acid; trans-(4-{[N-(2-{[N′-[3,5-bis(trifluoromethyl)benzyl-N′-(2-methyl-2H-tetrazol-5-yl)amino]methyl}-5-methyl-4-trifluoromethylphenyl)-N-ethylamino]methyl}cyclohexyl)acetic acid methanesulfonate; trans-(2R,4S)-2-(4-{4-[(3,5-bis-trifluoromethyl-benzyl)-(2-methyl-2H-tetrazol-5-yl)-amino]-2-ethyl-6-trifluoromethyl-3,4-dihydro-2H-quinoline-1-carbonyl}-cyclohexyl)-acetamide; methyl N-(3-cyano-5-trifluoromethylbenzyl)-6-(N′-cyclopentylmethyl-N′-ethylamino)indan-5-ylmethyl]-carbamate; methyl(3-cyano-5-trifluoromethylbenzyl)-[6-(N-cyclopentylmethyl-N-ethylamino)indan-5-ylmethyl]-carbamate; ethyl 4-((3,5-bis(trifluoromethyl)phenyl)(2-methyl-2H-tetrazol-5-yl)methyl)-2-ethyl-6-(trifluoromethyl)-3,4-dihydroquinoxaline-1(2H)-carboxylate; tert-butyl 5-(N-(3,5-bis(trifluoromethyl)benzyl)acetamido)-7-methyl-8-(trifluoromethyl)-2,3,4,5-tetrahydrobenzo[b]azepine-1-carboxylate, (3,5-bis-trifluoromethyl-benzyl)-[2-(cyclohexyl-methoxy-methyl)-5-trifluoromethyl-benzyl]-(2-methyl-2H-tetrazol-5-yl)-amine; 1-[1-(2-{[(3,5-bis-trifluoromethyl-benzyl)-(2-methyl-2H-tetrazol-5-yl)-amino]-methyl}-4-trifluoromethyl-phenyl)-2-methyl-propyl]-piperidine-4-carboxylic acid; (3,5-bis-trifluoromethyl-benzyl)-[2-(1-methoxy-cycloheptyl)-5-trifluoromethyl-benzyl]-(2-methyl-2H-tetrazol-5-yl)-amine; and (3,5-bis-trifluoromethyl-benzyl)-[2-(1-cyclohexyl-1-methoxy-ethyl)-5-trifluoromethyl-benzyl]-(2-methyl-2H-tetrazol-5-yl)-amine, the drugs disclosed in commonly owned U.S. patent application Ser. Nos. 09/918,127 and 10/066,091, the disclosures of both of which are incorporated herein by reference, and the drugs disclosed in the following patents and published applications, the disclosures of all of which are incorporated herein by reference: DE 19741400 A1; DE 19741399 A1; WO 9914215 A1; WO 9914174; DE 19709125 A1; DE 19704244 A1; DE 19704243 A1; EP 818448 A1; WO 9804528 A2; DE 19627431 A1; DE 19627430 A1; DE 19627419 A1; EP 796846 A1; DE 19832159; DE 818197; DE 19741051; WO 9941237 A1; WO 9914204 A1; JP 11049743; WO 0018721; WO 0018723; WO 0018724; WO 0017164; WO 0017165; WO 0017166; EP 992496; EP 987251; WO 9835937; JP 03221376; WO 04020393; WO 05095395; WO 05095409; WO 05100298; WO 05037796; WO 0509805; WO 03028727; WO 04039364; WO 04039453; WO 0633002; and U.S. Provisional Patent Application Nos. 60/781488 and 60/780993, both of which were filed on Mar. 10, 2006.

Thus, in one embodiment, the CETP inhibitor is selected from the group of compounds mentioned above. In another embodiment, the CETP inhibitor is selected from the group consisting of torcetrapib; (2R)-3-[[3-(4-chloro-3-ethylphenoxy)phenyl][[3-(1,1,2,2-tetrafluoroethoxy)phenyl]methyl]amino]-1,1,1-trifluoro-2-propanol; (2R, 4R, 4aS)-4-[amino-(3,5-bis-(trifluoromethyl-phenyl)-methyl]-2-ethyl-6-(trifluoromethyl)-3,4-dihydroquinoline-1-carboxylic acid isopropyl ester; trans-(2R,4S)-2-(4-{4-[(3,5-bis-trifluoromethyl-benzyl)-(2-methyl-2H-tetrazol-5-yl)-amino]-2-ethyl-6-trifluoromethyl-3,4-dihydro-2H-quinoline-1-carbonyl}-cyclohexyl)-acetamide; (3,5-bis-trifluoromethyl-benzyl)-[2-(cyclohexyl-methoxy-methyl)-5-trifluoromethyl-benzyl]-(2-methyl-2H-tetrazol-5-yl-amine; 1-[1-(2-{[(3,5-bis-trifluoromethyl-benzyl)-(2-methyl-2H-tetrazol-5-yl)-amino]-methyl}-4-trifluoromethyl-phenyl)-2-methyl-propyl]-piperidine-4-carboxylic acid; (3,5-bis-trifluoromethyl-benzyl)-[2-(1-methoxy-cycloheptyl)-5-trifluoromethyl-benzyl]-(2-methyl-2H-tetrazol-5-yl)-amine; (3,5-bis-trifluoromethyl-benzyl)-[2-(1-cyclohexyl-1-methoxy-ethyl)-5-trifluoromethyl-benzyl]-(2-methyl-2H-tetrazol-5-yl)-amine, and pharmaceutically acceptable forms thereof.

In another embodiment, the CETP inhibitor is torcetrapib.

In still another embodiment, the CETP inhibitor is (2R)-3-[[3-(4-chloro-3-ethylphenoxy)phenyl][[3-(1,1,2,2-tetrafluoroethoxy)phenyl]methyl]amino]-1,1,1-trifluoro-2-propanol.

In still another embodiment, the CETP inhibitor is trans-(2R,4S)-2-(4-{4-[(3 15-Bis-trifluoromethyl-benzyl)-(2-methyl-2H-tetrazol-5-yl)-amino]-2-ethyl-6-trifluoromethyl-3,4-dihydro-2H-quinoline-1-carbonyl}-cyclohexyl)-acetamide.

In another aspect, the drug is an inhibitor of cyclooxygenase-2 (COX-2). COX-2 inhibitors are nonsteroidal anti-inflammatory drugs that exhibit anti-inflammatory, analgesic and antipyretic effects. Preferably, the COX-2 inhibitor is a selective COX-2 inhibitor, meaning that the drug is able to inhibit COX-2 without significant inhibition of cyclooxygenase-1 (COX-1). Preferably, the COX-2 inhibitor has a potency such that the concentration of drug that inhibits 50% of COX-2 enzyme in an in vitro test (i.e., the IC₅₀ value) is less than about 10 μM, preferably less than 5 μM, more preferably less than 2 μM. In addition, it is also preferable that the COX-2 inhibitor be selective relative to COX-1. Thus, preferably, the ratio of the IC_{50,COX-2} to IC_{50,COX-1} ratio for the compound is less than 0.5, more preferably less than 0.3, and most preferably less than 0.2.

Specific examples of COX-2 inhibitors include 4-(5-(4-methylphenyl)-3-(trifluoromethyl)-1H-pyrazol-1-yl)benzenesulfonamide (celecoxib); 4-(5-methyl-3-phenylisoxazol-4-yl)benzenesulfonamide (valdecoxib); N-(4-(5-methyl-3-phenylisoxazol-4-yl)phenylsulfonyl)propionamide (paracoxb); sodium (S)-6,8-dichloro-2-(trifluoromethyl)-2H-chromene-3-carboxylate; sodium (S)-7-tert-butyl-6-chloro-2-(trifluoromethyl)-2H-chromene-3-carboxylate; 2-[(2-chloro-6-fluorophenyl)amino]-5-methyl benzeneacetic acid (lumiracoxib); 4-(3-(difluoromethyl)-5-(3-fluoro-4-methoxyphenyl)-1H-pyrazol-1-yl)benzenesulfonamide (deracoxib); 4-(4-(methylsulfonyl)phenyl)-3-phenylfuran-2(5H)-one (rofecoxib); 5-chloro-2-(6-methylpyridin-3-yl)-3-(4-(methylsulfonyl)phenyl)pyridine (etoricoxib); 2-(3,4-difluorophenyl)-4-(3-hydroxy-3-methylbutoxy)-5-(4-(methylsulfonyl)phenyl)pyridazin-3(2H)-one; (Z)-3-((3-chlorophenyl)(4-(methylsulfonyl)phenyl)methylene)-dihydrofuran-2(3H)-one; N-(2-(cyclohexyloxy)-4-nitrophenyl)methanesulfonamide; 4-Methyl-2-(3,4-dimethylphenyl)-1-(4-sulfamoyl-phenyl)-1H-pyrrole; 6-((5-(4-chlorobenzoyl)-1,4-dimethyl-1H-pyrrol-2-yl)methyl)pyridazin-3(2H)-one; 4-(4-cyclohexyl-2-methyloxazol-5-yl)-2-fluorobenzenesulfonamide (tilmacoxib); 2-(4-Ethoxyphenyl)-4-methyl-1-(4-sulfamoylphenyl)-1H-pyrrole; 4-hydroxy-2-methyl-N-(5-methyl-2-thiazolyl)-2H-1,2-benzothiazine-3-carboxamide-1,1-dioxide (meloxicam); 4-(4-chloro-5-(3-fluoro-4-methoxyphenyl)-1H-pyrazol-1-yl)benzenesulfonamide(cimicoxib), and pharmaceutically acceptable forms thereof; and the compounds disclosed in the following patents and published applications, the disclosures of which are incorporated herein by reference: U.S. Pat. No. 5,466,823, U.S. Pat. No. 5,633,272, U.S. Pat. No. 5,932,598, U.S. Pat. No. 6,034,256, U.S. Pat. No. 6,180,651, U.S. Pat. No. 5,908,858, U.S. Pat. No. 5,521,207, U.S. Pat. No. 5,691,374, WO 99/11605, WO 98/03484, and WO 00/24719. Preferably the COX-2 inhibitor is selected from the group consisting of celecoxib; valdecoxib; paracoxb; sodium (S)-6,8-dichloro-2-(trifluoromethyl)-2H-chromene-3-carboxylate; sodium (S)-7-tert-butyl-6-chloro-2-(trifluoromethyl)-2H-chromene-3-carboxylate; and pharmaceutically acceptable forms thereof. In one embodiment, the COX-2 inhibitor is celecoxib or pharmaceutically acceptable forms thereof.

### Processes for Forming Nanoparticles

The nanoparticles may be formed by any process that results in formation of nanoparticles comprising non-crystalline drug and an enteric polymer.

One process for forming nanoparticles is an emulsification process. In this process, the drug and enteric polymer are dissolved in an organic solvent that is immiscible with an aqueous solution in which the drug and enteric polymer are poorly soluble, forming an organic solution. Solvents suitable for forming the solution of dissolved drug and enteric polymers can be any compound or mixture of compounds in which the drug and the enteric polymer are mutually soluble and which is immiscible in the aqueous solution. As used herein, the term “immiscible” means that the organic solvent has a solubility in the aqueous solution of less than about 10 wt %, preferably less than about 5 wt %, and most preferably less than about 3 wt %. Preferably, the solvent is also volatile with a boiling point of 150° C. or less. In addition, the organic solvent preferably has relatively low toxicity. Exemplary organic solvents include methylene chloride, trichloroethylene, trichloro-trifluoroethylene, tetrachloroethane, trichloroethane, dichloroethane, dibromoethane, ethyl acetate, phenol, chloroform, toluene, xylene, ethyl-benzene, benzyl alcohol, creosol, methyl-ethyl ketone, methyl-isobutyl ketone, hexane, heptane, ether, and mixtures thereof. Preferred organic solvents are methylene chloride, ethyl acetate, benzyl alcohol, and mixtures thereof. The aqueous solution is preferably water.

Once the organic solution is formed, it is then mixed with the aqueous solution and homogenized to form an emulsion of fine droplets of the water immiscible solvent distributed throughout the aqueous phase. The volume ratio of organic solvent to aqueous solution used in the process will generally range from 1:100 (organic solvent:aqueous solution) to 2:3 (organic solvent:aqueous solution). Preferably, the organic solvent:aqueous solution volume ratio ranges from 1:9 to 1:2 (organic solvent:aqueous solution). The emulsion is generally formed by a two-step homogenization procedure. The solution of drug, enteric polymer and organic, solvent are first mixed with the aqueous solution using a rotor/stator or similar mixer to create a “pre-emulsion”. This mixture is then further processed with a high-pressure homogenizer that subjects the droplets to very high shear, creating a uniform emulsion of very small droplets. A portion of the organic solvent is then removed forming a suspension of the nanoparticles in the aqueous solution. Exemplary processes for removing the organic solvent include evaporation, extraction, diafiltration, pervaporation, vapor permeation, distillation, and filtration. Preferably, the organic solvent is removed to a level that is acceptable according to The International Committee on Harmonization (ICH) guidelines. Preferably, the concentration of organic solvent in the nanoparticle suspension is less than the solubility of the organic solvent in the aqueous solution. Even lower concentrations of organic solvent are preferred. Thus, the concentration of organic solvent in the nanoparticle suspension may be less than about 5 wt %, less than about 3 wt %, less than 1 wt %, and even less than 0.1 wt %.

An alternative process to form the nanoparticles is a precipitation process. In this process, the drug and enteric polymer are first dissolved in an organic solvent that is miscible with an aqueous solution in which the drug and enteric polymer are poorly soluble. The resulting organic solution is mixed with the aqueous solution causing nanoparticles to precipitate. Solvents suitable for forming the solution of dissolved drug and enteric polymers can be any compound or mixture of compounds in which the drug and the enteric polymer are mutually soluble and which is miscible in the aqueous solution. Preferably, the organic solvent is volatile with a boiling point of 150° C. or less. In addition, the organic solvent should have relatively low toxicity. Exemplary solvents include acetone, methanol, ethanol, tetrahydrofuran (THF), and dimethylsulfoxide (DMSO). Mixtures of solvents, such as 50% methanol and 50% acetone, can also be used, as can mixtures with water, so long as the enteric polymer and drug are sufficiently soluble to dissolve the drug and enteric polymer. Preferred solvents are methanol, acetone, and mixtures thereof.

The aqueous solution may be any compound or mixture of compounds in which the drug and enteric polymers are sufficiently insoluble so as to precipitate to form nanoparticles. The aqueous solution is preferably water.

The organic solvent solution and aqueous solution are combined under conditions that cause solids to precipitate as nanoparticles. The mixing can be by addition of a bolus or stream of solvent solution to a stirring container of the aqueous solution. Alternately a stream or jet of solvent solution can be mixed with a moving stream of the aqueous solution. In either case, the precipitation results in the formation of a suspension of nanoparticles in the aqueous solution.

For the precipitation process, the amount of drug and polymer in the solvent solution depends on the solubility of each in the solvent and the desired ratios of drug to polymer in the resulting nanoparticles. The solution may comprise from about 0.1 wt % to about 20 wt % dissolved solids. A dissolved solids content of from about 0.5 wt % to 10 wt % is preferred.

The organic solvent:aqueous solution volume ratio should be selected such that there is sufficient aqueous solution in the nanoparticle suspension that the nanoparticles solidify and do not rapidly agglomerate. However, too much aqueous solution will result in a very dilute suspension of nanoparticles, which may require further processing for ultimate use. Generally, the organic solvent:aqueous solution volume ratio should be at least 1:100, but generally should be less than 1:2 (organic solvent:aqueous solution). Preferably, the organic solvent:aqueous solution volume ratio ranges from about 1:20 to about 1:3.

Once the nanoparticle suspension is made, a portion of the organic solvent may be removed from the suspension using methods known in the art. Exemplary processes for removing the solvent include evaporation, extraction, diafiltration, pervaporation, vapor permeation, distillation, and filtration. Preferably, the organic solvent is removed to a level that is acceptable according to ICH guidelines. Thus, the concentration of organic solvent in the nanoparticle suspension may be less than about 10 wt %, less than about 5 wt %, less than about 3 wt %, less than 1 wt %, and even less than 0.1 wt %.

### Formation of Compositions

The compositions of the present invention comprise nanoparticles comprising a drug and enteric polymer, and casein. The casein can be formulated with the nanoparticles either during the process used to form the nanoparticles or after the nanoparticles are formed.

In one embodiment, the casein is formulated with the nanoparticles during the nanoparticle-formation process. In this embodiment, the casein may be considered to be part of the nanoparticles. For the emulsion and precipitation processes described above, the casein can be either added to the organic solution comprising the drug and enteric polymer or added to the aqueous solution, in which the drug and polymer are poorly soluble. In a preferred embodiment, the casein is added to the aqueous solution. Formulating the casein in the aqueous solution is advantageous as it allows the casein to help reduce or eliminate flocculation or aggregation of the nanoparticles once they are formed.

Thus, in one embodiment, the compositions of the present invention are formed by the process comprising (a) forming an organic solution comprising a poorly water soluble drug and an enteric polymer dissolved in a water-immiscible solvent, (b) forming an aqueous solution comprising casein, (c) mixing the organic solution and the aqueous solution to form an emulsion, and (d) removing the water-immiscible solvent from the emulsion to form an aqueous suspension comprising nanoparticles comprising the poorly water soluble drug and the enteric polymer, and casein.

In another embodiment, the compositions of the present invention are formed by the process comprising (a) forming an organic solution comprising a poorly water soluble drug and an enteric polymer dissolved in a water-miscible solvent, (b) forming an aqueous solution comprising casein, (c) mixing the organic solution and the aqueous solution to form an aqueous suspension comprising nanoparticles comprising the poorly water soluble drug and the enteric polymer, and casein.

In another embodiment, the casein is formulated with the nanoparticles after the nanoparticles have been formed. This has advantages when the process for removing the solvent from the nanoparticle suspension would also remove the casein (e.g., diafiltration). This embodiment is also preferred when processes are used to increase the concentration of nanoparticles in the suspension. Generally, in this embodiment, casein is administered to the suspension containing the nanoparticles. Note that when the nanoparticles are suspended in an aqueous solution, the casein may not completely dissolve in the water. As discussed above, casein often forms micelles when added to water. In such instances, the casein may be present in the form of micelles.

In still another embodiment, a process for forming nanoparticles, comprises: (a) forming an organic solution comprising a poorly water soluble drug and an enteric polymer dissolved in a solvent, wherein (i) the drug has a solubility in water of less than 5 mg/ml over the pH range of 6.5 to 7.5, and (ii) a mass ratio of the poorly water soluble drug to the enteric polymer is less than 9:1; (b) forming an aqueous solution; (c) mixing the organic solution with the aqueous solution to form a first mixture; (d) removing the solvent from the first mixture to form a suspension comprising the nanoparticles and the aqueous solution, wherein (i) the nanoparticles have an average size of less than 500 nm, and (ii) at least 90 wt % of the drug in the nanoparticles is non-crystalline; and (e) adding casein to either the aqueous solution of step (b) or to the suspension of step (d), wherein a mass ratio of the casein to the poorly water soluble drug and the enteric polymer is at least 1:20. In one embodiment, the process comprises the additional step (f) removing liquid from the suspension to form a solid composition comprising the nanoparticles and the casein.

A variety of processes may be used to form solid compositions comprising nanoparticles comprising a poorly water soluble drug and an enteric polymer, and casein. Essentially any process that removes the liquid from the suspension may be used to form a solid composition, provided the process does not affect the properties of the nanoparticles or casein. Exemplary processes include spray drying, spray coating, spray layering, lyophylization, evaporation, vacuum evaporation, and filtration. A preferred process is spray drying, as described in the Examples. One or more processes may be combined to remove the liquid from the nanoparticle/casein suspension and yield a solid composition. For example, a portion of the organic solvent and aqueous solution may be removed by filtration to concentrate the nanoparticles, followed by spray-drying to remove most of the remaining liquids, followed by a further drying step such as tray-drying.

Once the solid composition is formed, it may be desirable to form small particles of the solid composition, as discussed above. Some of the processes described above, such as spray drying, will typically produce small particles of the solid composition. Other processes used to form the solid composition may result in larger particles, sheets, flakes, or other forms of the solid composition. Thus, the particle size of the solid composition may be adjusted using various techniques known in the art, such as through the use of grinders and, mills. See, for example, Remington: The Science and Practice of Pharmacy, 20^{th} Edition (2000).

### Resuspendability

In one embodiment, the solid compositions of the present invention result in improved resuspendability of the nanoparticles relative to surfactant-based and polymer-based stabilizers. The term “resuspendability” as used herein means the ability of the solid material, when administered to an aqueous use environment, to form a nanoparticle suspension.

The ability of the solid composition to resuspend nanoparticles when administered to an aqueous solution can be determined using the following procedures. In the first procedure, the average particle size of the re-suspended material is determined as follows. The solid composition is added to an aqueous solution, such as water, PBS, or MFD solution, to form a suspension. A sample of the solid composition is added to water at ambient temperature such that the concentration of solids is less than about 1 mg/mL. The average particle size of the nanoparticles formed during this (re)suspension is then determined by dynamic light scattering (DLS) techniques. A solid composition is said to provide good resuspendability if, upon administration to an aqueous solution, the average particle size as determined by DLS techniques is at least 50% and no more than 200% the average particle size of the nanoparticles prior to recovery of the solid composition. Preferably, the formulation provides an average particle size that is at least 67% and no more than 150% the average particle size prior to recovery of the solid composition. Even more preferably, the formulation provides an average particle size that is at least 75% and no more than 133% the average particle size prior to recovery of the solid composition.

The second procedure is known as a filter potency test. In this test the concentration of drug after passing the suspension of the nanoparticles through a filter is determined. The solid composition is added to an aqueous solution as described above. The concentration of drug in the so-formed suspension is then determined using standard techniques, such as by high-performance liquid chromatography (HPLC). Next, the suspension is filtered through a filter, and the concentration of drug in the filtered sample is determined via standard techniques. A loss in potency after filtering a sample through a filter is an indication that the nanoparticles in the sample are larger than the filter pore size. Exemplary filters that can be used in this test include a 1-μm glass fiber filter, a 0.45-μm syringe filter, and a 0.2-μm syringe filter. One skilled in the art will understand that the pore size of the filter should be selected to ensure the nanoparticles are not retained on the filter. Generally, the pore size of filter and the range of nanoparticle average diameters are given as follows:

| Filter Pore Size (μm) | Suitable Range of Nanoparticle Diameters (nm) |
|---|---|
| 1 | >250 |
| 0.45 | 150 to 300 |
| 0.2 | <200 |

A solid composition is said to provide good resuspendability if the ratio of the concentration of drug in the filtered sample is at least 60% the concentration of drug in the unfiltered sample. Preferably, the concentration of drug in the filtered sample is at least 70% the concentration of drug in the unfiltered sample. Most preferably, the concentration of drug in the filtered sample is at least 80% the concentration of drug in the unfiltered sample.

In an especially preferred embodiment, a composition provides good resuspendability in both of the tests described above.

### Dosage Forms

The compositions of the present invention may be administered using any known dosage form. The nanoparticles may be formulated for administration via oral, subdermal, intranasal, buccal, intrathecal, ocular, intraaural, subcutaneous spaces, intraarticular, vaginal tract, arterial and venous blood vessels, pulmonary tract or intramuscular tissue of an animal, such as a mammal and particularly a human. Oral dosage forms include: powders or granules; tablets; chewable tablets; capsules; unit dose packets, sometimes referred to in the art as “sachets” or “oral powders for constitution” (OPC); syrups; and suspensions. Parenteral dosage forms include reconstitutable powders or suspensions. Topical dosage forms include creams, pastes, suspensions, powders, foams and gels. Ocular dosage forms include suspensions, powders, gels, creams, pastes, solid inserts and implants.

In one embodiment, the compositions of the present invention are capable of improving the concentration of dissolved drug in a use environment relative to a control composition consisting essentially of the drug alone without any enteric polymer or casein. In order to determine concentration enhancement in vitro, the amount of “free” drug, or solvated drug is measured. By “free” drug is meant drug which is in the form of dissolved drug or present in micelles, but which is not in the nanoparticles or any solid particles larger than 500 nm, such as precipitate. A composition of the invention provides concentration enhancement if, when administered to an aqueous use environment, it provides a free drug concentration that is at least 1.25-fold the free drug concentration provided by the control composition. Preferably, the free drug concentration provided by the compositions of the invention are at least about 1.5-fold, more preferably at least about 2-fold, and most preferably at least about 3-fold that provided by the control composition.

Alternatively, the compositions of the present invention, when dosed orally to a human or other animal, provide an AUC in drug concentration in the blood plasma or serum (or relative bioavailability) that is at least 1.25-fold that observed in comparison to the control composition. Preferably, the blood AUC is at least about 2-fold, more preferably at least about 3-fold, even more preferably at least about 4-fold, still more preferably at least about 6-fold, yet more preferably at least about 10-fold, and most preferably at least about 20-fold that of the control composition. The determination of AUCs is a well-known procedure and is described, for example, in Welling, “Pharmacokinetics Processes and Mathematics,” ACS Monograph 185 (1986).

Alternatively, the compositions of the present invention, when dosed orally to a human or other animal, provide a maximum drug concentration in the blood plasma or serum (Cₘₐₓ) that is at least 1.25-fold that observed in comparison to the control composition. Preferably, the Cₘₐₓ is at least about 2-fold, more preferably at least about 3-fold, even more preferably at least about 4-fold, still more preferably at least about 6-fold, yet more preferably at least about 10-fold, and most preferably at least about 20-fold that of the control composition. Thus, compositions that meet the in vitro or in vivo performance criteria, or both, are considered to be within the scope of the invention.

Without further elaboration, it is believed that one of ordinary skill in the art can, using the foregoing description, utilize the present invention to its fullest extent. Therefore, the following specific embodiments are to be construed as merely illustrative and not restrictive of the scope of the invention. Those of ordinary skill in the art will understand that variations of the conditions and processes of the following examples can be used.

### EXAMPLES

### Drugs Used in Examples

In the following examples, Drug 1 was (2R)-3-[[3-(4-chloro-3-ethylphenoxy)phenyl][[3-(1,1,2,2-tetrafluoroethoxy)phenyl]methyl]amino]-1,1,1-trifluoro-2-propanol, having the structure:

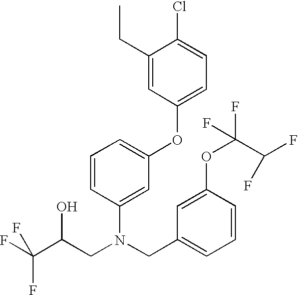

Drug 1 has a solubility in PBS of less than 0.1 μg/mL, and a Clog P value of 9.8. The Tₘ of Drug 1 is 10° C., and the T_{g} was determined by DSC analysis to be −16° C.

### Excipients Used in the Examples

The following enteric polymers were used in the examples: hydroxypropyl methylcellulose acetate succinate (HPMCAS-L, AQOAT-L from Shin Etsu, Tokyo, Japan), and carboxymethyl ethylcellulose (CMEC, available from Freund Industrial Co., Ltd., Japan).

Sodium caseinate was obtained from several sources: (1) Spectrum Chemicals, Gardena, Calif., (2) American Casein Company, Burlington, N.J., and (3) Sigma Chemicals, St Louis, Mo.

### Example 1

The nanoparticles of Example 1 were made containing Drug 1, hydroxypropyl methylcellulose acetate succinate (HPMCAS-L, AQOAT-L from Shin Etsu, Tokyo, Japan), and casein. First, 150 mg Drug 1 and 150 mg HPMCAS were dissolved in 5 mL 3:1 ethyl acetate:methylene chloride to form an organic solution. Next, 100 mg sodium caseinate was added to 20 mL deionized water to form an aqueous solution. The organic solution was then poured into the aqueous solution and emulsified for 3 min using a Kinematica Polytron 3100 rotor/stator (Kinematica AG, Lucerne, Switzerland) at 10,000 rpm (high-shear mixing). The solution was further emulsified using a Microfluidizer (Microfluidics [Newton, Mass.] model M-110S F12Y with ice bath and cooling coil), for 6 minutes (high-pressure homogenization). The ethyl acetate and methylene chloride were removed from the emulsion using a rotary evaporator to a combined concentration of less than about 3 wt %, resulting in an aqueous suspension of nanoparticles, with a mass ratio of 37.5:37.5:25 Drug 1:HPMCAS:caseinate.

### Light Scattering Analysis

The particle size of the nanoparticles in the aqueous suspension was determined using dynamic light scattering (DLS) as follows. First, the aqueous suspension was filtered using a 1 μm glass membrane filter (Anotop filter, Whatman), and poured into a cuvette. Light-scattering was measured using a Brookhaven Instruments (Holtsville, N.Y.) BI-200SM particle size analyzer with a BI-9000AT correlator. The sums of exponentials from the autocorrelation functions are analyzed to extract size distributions from the samples, and the size is reported as the cumulant value. The average diameter was found to be 100 nm, with a polydispersity of 0.25.

The aqueous suspension of Example 1 was allowed to stand unmixed for 24 hours at ambient conditions to measure stability. DLS analysis showed that the average cumulant diameter of the nanoparticles in suspension was 119 nm, with a polydispersity of 0.26. These results demonstrate that the nanoparticles of Example 1 in suspension were stable during storage with no significant particle agglomeration.

### Isolation of Solid Compositions

The nanoparticle suspension of Example 1 was spray-dried as follows. The suspension was added to a reservoir and pumped to a two fluid nozzle located in a spray-drying chamber, using an HPLC pump (model 515, Waters Corp., Milford, Mass.) at a flow rate of about 0.15 g/min. The spray-drying chamber consisted of two sections: a straight-side section (top), and a cone section (bottom). The top of the straight-side section was equipped with a spray-solution inlet. The spray solution was sprayed through the spray-solution inlet using the two-fluid nozzle, into the straight-side section of the spray-drying chamber. The straight-side section had a diameter of 10 cm and a length of 19 cm.

Drying gas (nitrogen) entered the cone section through a drying-gas inlet at a flow of about 1.0 SCFM and an inlet temperature of about 120° C. The flow rate of drying gas and spray solution were selected such that the atomized spray solution was sufficiently dry by the time it reached the walls of the spray-drying chamber that it did not stick to the walls. The diameter of the cone section at the top was 10 cm, and the distance from the top of the cone section to the bottom was 19 cm. At the bottom of the cone section was a 4.7-cm diameter outlet port, fitted with a 0.8 μm nylon filter (Magna, GE Osmonics, Minnetonka, Minn.) supported by a metal screen. The spray dried composition was collected on the filter, and evaporated solvent and drying gas were removed from the spray-drying chamber through the outlet port.

### Nanoparticle Resuspension

The solid composition of Example 1 was resuspended by adding 8.7 mg of sample to 2 mL deionized water. DLS analysis showed that the average cumulant diameter of the nanoparticle suspension was 144 nm, with a polydispersity of 0.44. This demonstrates that a small particle size was maintained after isolation of the solid composition of Example 1, followed by resuspension.

### Filter Potency

Filter potency was used to characterize the resuspended nanoparticles of Example 1. First, a 50 μL sample of the aqueous nanoparticle suspension was added to 1 mL methanol, and the concentration of drug in solution was analyzed by HPLC. Next, the suspension was filtered using a 0.45 μm filter and diluted in methanol for HPLC analysis.

Potencies of the nanoparticle suspensions are shown in Table 2. The results in Table 2 show that 82% of the nanoparticle suspension potency is maintained following filtration of Example 1 using a 0.45 μm filter. This indicates that the nanoparticles in suspension remain small and unagglomerated.

TABLE 2

| | | Potency | |
|---|---|---|---|
| | Potency | 0.45 μm | |
| | Unfiltered | filtered | Potency |
| Sample | (mg/mL) | (mg/mL) | Retained (%) |
| Example 1 | 1.7 | 1.4 | 82 |

### Example 2

For Example 2, nanoparticles containing Drug 1 were prepared using a precipitation method as follows. First, a water-miscible organic solution was formed by dissolving 200 mg Drug 1 and 373.2 mg HPMCAS-L in 37 mL methanol. To form the nanoparticles, the stem of a glass funnel containing the organic solution was inserted under the surface of an aqueous solution consisting of 343 mL of filtered water, and delivered into the stirring vortex all at once, rapidly forming nanoparticles. The methanol was removed using a rotary evaporator to a concentration of less than about 0.1 wt %, resulting in an aqueous suspension of nanoparticles. DLS analysis showed that the average cumulant diameter of the nanoparticles in suspension was 109 nm, with a polydispersity of 0.26.

The aqueous suspension was concentrated using tangential flow filtration with a Millipore Biomax® 300 50 cm² polyethersulfone membrane (available from Millipore Corp., Billerica, Mass.). The feed solution, consisting of about 345 mL aqueous nanoparticle suspension, was concentrated to 24 mL final volume.

To form an aqueous suspension of the present invention, sodium caseinate was added to this concentrated suspension, resulting in a nanoparticle suspension consisting of 26.2:48.8:25 Drug 1:HPMCAS-LF:casein.

### Isolation of Solid Compositions

The nanoparticle suspension of Example 2 was spray-dried using the procedures described in Example 1, resulting in the formation of a solid composition of the invention.

### Nanoparticle Resuspension

The solid composition of Example 2 was resuspended by adding about 5 mg/mL sample to deionized water. DLS analysis showed that the average cumulant diameter of the resuspended nanoparticles was 157 nm, with a polydispersity of 0.26. This demonstrates that a small particle size can be maintained after isolation of the solid composition, followed by resuspension.

### Filter Potency

A filter potency test was used to characterize resuspended nanoparticles of Example 2. A 50 μL sample of the aqueous nanoparticle resuspension of Example 2 was added to 1 mL methanol, and the concentration of drug in solution was analyzed by HPLC. Next, the suspension was filtered using a 0.2 μm filter, and diluted in methanol for HPLC analysis.

Potencies of the nanoparticle suspensions are shown in Table 3. The results in Table 3 show that 94% of the nanoparticle suspension potency is maintained following filtration by a 0.2 μm filter. This indicates that most of the nanoparticles in suspension remain small and unagglomerated.

TABLE 3

| | | Potency | |
|---|---|---|---|
| | Potency | 0.2 μm | |
| | Unfiltered | filtered | Potency |
| Sample | (mg/mL) | (mg/mL) | Retained (%) |
| Example 2 | 2.47 | 2.33 | 94 |

### Example 3

Nanoparticles containing Drug 1 and the enteric polymer carboxymethyl ethylcellulose (CMEC, available from Freund Industrial Co., Ltd., Japan) were prepared using the procedure outlined in Example 2 with the following exceptions. The water-miscible organic solution was formed by dissolving 93 mg Drug 1 and 181.2 mg CMEC in 20 mL methanol. The aqueous solution consisted of 180 mL of filtered water. The organic solution and aqueous solutions were then mixed rapidly to form nanoparticles. The methanol was removed using rotary evaporation to a concentration of less than 0.5 wt %, resulting in a nanoparticle suspension consisting of 34:66 (wt:wt) Drug 1:CMEC. DLS analysis showed that the average cumulant diameter of the nanoparticles in suspension was 110 nm, with a polydispersity of 0.39. The aqueous suspension was concentrated as described in Example 2.

To form an aqueous suspension of the present invention, sodium caseinate was added to this concentrated suspension, resulting in a nanoparticle suspension consisting of 25.5:49.5:25 Drug 1:CMEC:casein.

### Isolation of Solid Compositions

The nanoparticle suspension of Example 3 was spray-dried using the procedures described in Example 1, resulting in the formation of a solid composition of the invention.

### Nanoparticle Resuspension

The solid composition of Example 3 was resuspended by adding 38 mg of sample to 2 mL deionized water. DLS analysis showed that the average cumulant diameter of the nanoparticle suspension was 165 nm, with a polydispersity of 0.38. This demonstrates that a small particle size can be maintained after isolation of the solid composition, followed by resuspension.

The terms and expressions which have been employed in the foregoing specification are used therein as terms of description and not of limitation, and there is no intention in the use of such terms and expressions of excluding equivalents of the features shown and described or portions thereof, it being recognized that the scope of the invention is defined and limited only by the claims which follow.

1. A solid pharmaceutical composition comprising:
(a) nanoparticles comprising a poorly water soluble drug and an enteric polymer, wherein
(i) said poorly water soluble drug has a water solubility of less than 5 mg/mL at a pH of 6.5 to 7.5,
(ii) at least 90 wt % of said drug in said nanoparticles is in a noncrystalline form,
(iii) said nanoparticles have an average diameter of less than 500 nm, and
(iv) the mass ratio of said poorly water soluble drug to said enteric polymer is less than 9:1, and
(b) casein or a pharmaceutically acceptable form thereof
wherein the mass ratio of (1) said casein to (2) the combined mass of said poorly water soluble drug and said enteric polymer is at least 1:20.

2. The composition of claim 1 wherein said mass ratio of (1) said casein to (2) the combined mass of said poorly water soluble drug and said enteric polymer is at least 1:10.

3. The composition of claim 1 wherein said poorly water soluble drug, said enteric polymer, and said casein constitute at least 70 wt % of said composition.

4. The composition of claim 1 wherein said poorly water soluble drug, said enteric polymer, and said casein constitute at least 80 wt % of said composition.

5. The composition of claim 1 wherein said composition consists essentially of said poorly water soluble drug, said enteric polymer, and said casein.

6. The composition of claim 1 wherein the mass ratio of said poorly water soluble drug to said enteric polymer is less than 4:1.

7. The composition of claim 1 wherein the mass ratio of said poorly water soluble drug to said enteric polymer ranges from 1:19 to 3:1.

8. The composition of claim 1 wherein said enteric polymer is selected from the group consisting of hydroxypropyl methyl cellulose acetate succinate, hydroxypropyl methyl cellulose phthalate, carboxymethyl ethylcellulose, cellulose acetate phthalate, cellulose acetate succinate, hydroxypropyl methyl cellulose acetate phthalate, cellulose i acetate trimellitate, hydroxypropyl methyl cellulose acetate trimellitate, polyvinyl acetate phthalate, vinyl acetate-maleic anhydride copolymer, polyacrylates, methyl acrylate-methacrylic acid copolymers, ethyl acrylate-methacrylic acid copolymers, styrene-maleic acid copolymers, shellac, and mixtures thereof.

9. The composition of claim 1 wherein said enteric polymer is selected from the group consisting of hydroxypropyl methyl cellulose acetate succinate, carboxymethylethyl cellulose, hydroxypropyl methyl cellulose phthalate, cellulose acetate phthalate, cellulose acetate trimellitate, methyl acrylate-methacrylic acid copolymers, ethyl acrylate-methacrylic acid copolymers, and mixtures thereof.

10. The composition of claim 1 wherein said casein is selected from the group consisting of αₛi-casein, αₛ₂-casein, β-casein, κ-casein, vegetable casein, sodium caseinate, calcium caseinate, potassium caseinate, ammonium caseinate, and mixtures thereof.

11. The composition of claim 1 wherein said nanoparticles further comprise a surface stabilizer.

12. The composition of claim 11 wherein said poorly water soluble drug, said enteric polymer, said surface stabilizer, and said casein constitute at least 90 wt % of said composition.

13. The composition of claim 12 wherein said composition consists essentially of said poorly water soluble drug, said enteric polymer, said surface stabilizer, and said casein.

14. The composition of claim 11 wherein said surface stabilizer is selected from the group consisting of casein, caseinates, polyvinyl pyrrolidone, polyoxyethylene alkyl ethers, polyoxyethylene stearates, polyoxyethylene castor oil derivatives, poly(ethylene oxide-propylene oxide), tragacanth, gelatin, polyethylene glycol, bile salts, phospholipids, sodium dodecylsulfate, benzalkonium chloride, sorbitan esters, polyoxyethylene alkyl ethers, polyoxyethylene castor oil derivatives, polyoxyethylene sorbitan fatty acid esters, polyoxyethylene stearates, triethanolamine, sodium docusate, sodium stearyl fumarate, sodium cyclamate, and mixtures and pharmaceutically acceptable forms thereof.

15. The composition of claim 1 wherein said composition comprises 1 to 60 wt % of said poorly aqueous soluble drug, 10 to 80 wt % of said enteric polymer, and 10 to 50 wt % of said casein.

16. The composition of claim 1 wherein said poorly water soluble drug and said enteric polymer are present in said nanoparticle in the form of a solid solution.

17. The composition of claim 1 wherein said nanoparticles are encapsulated within said casein.

18. The composition of claim 1 wherein said nanoparticles further comprise casein.

19. The composition of claim 1 wherein said solid composition further comprises water.

20. The composition of claim 1 wherein said poorly water soluble drug is a cholesteryl ester transfer protein inhibitor.

21. The composition of claim 20 wherein said cholesteryl ester transfer protein inhibitor is selected from the group consisting of torcetrapib; (2R)-3-[[3-(4-chloro-3-ethylphenoxy)phenyl][[3-(1,1,2,2-tetrafluoroethoxy)phenyl]methyl]amino]-1,1,1-trifluoro-2-propanol; (2R, 4R, 4aS)-4-[amino-(3,5-bis-(trifluoromethyl-phenyl)-methyl]-2-ethyl-6-(trifluoromethyl)-3,4-dihydroquinoline-1-carboxylic acid isopropyl ester; trans-(2R,4S)-2-(4-{4-[(3,5-Bis-trifluoromethyl-benzyl)-(2-methyl-2H-tetrazol-5-yl)-amino]-2-ethyl-6-trifluoromethyl-3,4-dihydro-2H-quinoline-1-carbonyl}-cyclohexyl)-acetamide; (3,5-Bis-trifluoromethyl-benzyl)-[2-(cyclohexyl-methoxy-methyl)-5-trifluoromethyl-benzyl]-(2-methyl-2H-tetrazol-5-yl)-amine; 1-[1-(2-{[(3,5-Bis-trifluoromethyl-benzyl)-(2-methyl-2H-tetrazol-5-yl)-amino]-methyl}-4-trifluoromethyl-phenyl)-2-methyl-propyl]-piperidine-4-carboxylic acid; (3,5-Bis-trifluoromethyl-benzyl)-[2-(1-methoxy-cycloheptyl)-5-trifluoromethyl-benzyl]-(2-methyl-2H-tetrazol-5-yl)-amine; (3,5-Bis-trifluoromethyl-benzyl)-[2-(1-cyclohexyl-1-methoxy-ethyl)-5-trifluoromethyl-benzyl]-(2-methyl-2H-tetrazol-5-yl)-amine; and pharmaceutically acceptable forms thereof.

22. The composition of claim 1 wherein said poorly water soluble drug is an inhibitor of cyclooxygenase-2.

23. The composition of claim 22 wherein said inhibitor of cyclooxygenase-2 is selected from the group consisting of celecoxib; valdecoxib; paracoxb; sodium (S)-6,8-dichloro-2-(trifluoromethyl)-2H-chromene-3-carboxylate; sodium (S)-7-tert-butyl-6-chloro-2-(trifluoromethyl)-2H-chromene-3-carboxylate; and pharmaceutically acceptable forms thereof.

24. A pharmaceutical composition comprising an
aqueous suspension, said aqueous suspension comprising:
(a) nanoparticles comprising a poorly water soluble drug and an enteric polymer, wherein
(i) said poorly water soluble drug has a water solubility of less than 5 mg/mL at a pH of 6.5 to 7.5,
(ii) at least 90 wt % of said drug in said nanoparticles is in a noncrystalline form,
(iii) said nanoparticles have an average diameter of less than 500 nm,
(iv) said poorly water soluble drug and said enteric polymer together constitute at least 60 wt % of said nanoparticles, and
(v) the mass ratio of said poorly water soluble drug to said enteric polymer is less than 9:1;
(b) casein or a pharmaceutically acceptable form thereof; and
(c) water.

25. The composition of claim 24 wherein said nanoparticles have an average diameter of less than 300 nm.

26. The composition of claim 24 wherein said nanoparticles and said casein collectively are present in said suspension at a concentration of at least 1 mg/mL.

27. The composition of claim 24 wherein said casein is associated with the surfaces of said nanoparticles.

28-34. (canceled)
